(19) United States
(12) Patent Application Publication
(10) Pub. No.: US 2022/0135540 A1
(43) Pub. Date: May 5, 2022

(54) POLYMORPHS OF AN FXR AGONIST

(71) Applicant: Eli Lilly and Company, Indianapolis, IN (US)

(72) Inventors: Stephanie Ann SWEETANA, Indianapolis, IN (US); Steven Jerald Sproull, Indianapolis, IN (US); Paul K. Owens, Indianapolis, IN (US)

(21) Appl. No.: 17/450,966

(22) Filed: Oct. 14, 2021

Related U.S. Application Data
(60) Provisional application No. 63/092,423, filed on Oct. 15, 2020.

(51) Int. Cl. C07D 401/14; A61P 1/16

(52) U.S. Cl. CPC C07D 401/14; A61P 1/16

(57) ABSTRACT

Provided herein are polymorphs of 6-(4-((5-cyclopropyl-3-(2,6-dichlorophenyl)isoxazol-4-yl)methoxy)piperidin-1-yl)-1-methyl-1H-indole-3-carboxylic acid, compositions thereof, methods of preparation thereof, and methods of use thereof.

### CROSS-REFERENCE TO RELATED APPLICATIONS

This application claims priority to and the benefit of U.S. Provisional Application No. 63/092,423, filed on Oct. 15, 2020, the disclosure of which is incorporated herein by reference in its entirety.

### FIELD

Provided herein are polymorphs of 6-(4-((5-cyclopropyl-3-(2,6-dichlorophenyl)isoxazol-4-yl)methoxy)piperidin-1-yl)-1-methyl-1H-indole-3-carboxylic acid, compositions thereof, methods of preparation thereof, and methods of use thereof.

### BACKGROUND

Therapeutic agents that function as farnesoid X receptor (FXR) agonists have the potential to remedy or improve the lives of patients in need of treatment of liver disorders such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). U.S. Pat. No. 8,153,624, the content of which is incorporated herein by reference in its entirety, discloses 6-(4-((5-cyclopropyl-3-(2,6-dichlorophenyl)isoxazol-4-yl)methoxy)piperidin-1-yl)-1-methyl-1H-indole-3-carboxylic acid (designated herein as Compound I), which has the structure shown below.

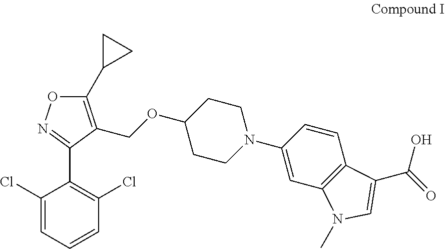 [SMILES: Cn1cc(C(=O)O)c2ccc(N3CCC(OCc4c(-c5c(Cl)cccc5Cl)noc4C4CC4)CC3)cc21]

Compound I is a potent FXR agonist being developed as a therapeutic for liver disorders. To move a drug candidate such as Compound I to a viable pharmaceutical product, it can be important to understand whether the drug candidate has polymorphic forms, as well as the relative stability and interconversions of these forms under conditions likely to be encountered upon large-scale production, transportation, storage, and pre-usage preparation. The ability to control and produce a stable polymorph with a robust manufacturing process can be key for regulatory approval and marketing. Large scale production processes for high purity Compound I can be improved by use of particular polymorphic forms. Accordingly, there is a need for various new polymorphic forms of Compound I with different chemical and physical stabilities, and compositions and uses of the same.

### BRIEF SUMMARY

In one aspect, provided herein are polymorphs of Compound I.

In another aspect, provided herein are methods of preparing polymorphs of Compound I.

In another aspect, provided herein are compositions comprising polymorphs of Compound I.

In another aspect, provided herein are methods of treating a subject in need of treatment of liver disorders using polymorphs of Compound I. Also provided is use of polymorphs of Compound I in the manufacture of a medicament for treating liver disorders.

### DESCRIPTION OF THE DRAWINGS

FIG. 1A shows an X-ray powder diffraction (XRPD) pattern of polymorphic Form I of Compound I.

FIG. 1B shows a differential scanning calorimetry (DSC) graph of polymorphic Form I of Compound I.

FIG. 1C shows a thermogravimetric analysis (TGA) graph of polymorphic Form I of Compound I.

FIG. 1D shows a moisture sorption analysis (MSA) graph of polymorphic Form I of Compound I.

FIG. 2A shows an XRPD pattern of polymorphic Form II of Compound I.

FIG. 2B shows a DSC graph of polymorphic Form II of Compound I.

FIG. 2C shows a TGA graph of polymorphic Form II of Compound I.

FIG. 2D shows a MSA graph of polymorphic Form II of Compound I.

FIG. 3A shows an XRPD pattern of polymorphic Form III of Compound I.

FIG. 3B shows a DSC graph of polymorphic Form III of Compound I.

FIG. 4A shows an XRPD pattern of polymorphic Form IV of Compound I.

FIG. 4B shows a DSC graph of polymorphic Form IV of Compound I.

FIG. 5A shows an XRPD pattern of polymorphic Form V of Compound I.

FIG. 5B shows a DSC graph of polymorphic Form V of Compound I.

FIG. 6A shows an XRPD pattern of polymorphic Form VI of Compound I.

FIG. 6B shows a DSC graph of polymorphic Form VI of Compound I.

FIG. 7A shows an XRPD pattern of polymorphic Form VII of Compound I.

FIG. 7B shows a DSC graph of polymorphic Form VII of Compound I.

### DETAILED DESCRIPTION

### Definitions

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in connection with a value, contemplate a variation within ±15%, within ±10%, within ±5%, within ±4%, within ±3%, within ±2%, within ±1%, or within ±0.5% of the specified value. Reference to “about” a value or parameter herein includes (and describes) embodiments that are directed to that value or parameter per se. For example, description referring to “about X” includes description of “X”.

As used herein, the term “polymorph” or “polymorphic form” refers to a crystalline form of a compound. Different polymorphs may have different physical properties such as, for example, melting temperatures, heats of fusion, solubilities, dissolution rates, and/or vibrational spectra as a result of the arrangement or conformation of the molecules or ions in the crystal lattice. The differences in physical properties exhibited by polymorphs may affect pharmaceutical parameters, such as storage stability, compressibility, density (important in formulation and product manufacturing), and dissolution rate (an important factor in bioavailability).

As used herein, the term “pharmaceutically acceptable carrier,” and cognates thereof, refers to adjuvants, binders, diluents, etc. known to the skilled artisan that are suitable for administration to an individual (e.g., a mammal or non-mammal). Combinations of two or more carriers are also contemplated. The pharmaceutically acceptable carrier(s) and any additional components, as described herein, should be compatible for use in the intended route of administration (e.g., oral, parenteral) for a particular dosage form, as would be recognized by the skilled artisan.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

The term “subject” refers to an animal, including, but are not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, the term “therapeutically effective amount” refers to an amount of a compound or composition sufficient to treat a specified disorder, condition or disease such as to ameliorate, to palliate, to lessen, and/or to delay one or more of its symptoms.

As used herein, the term “substantially as shown in” when referring, for example, to an XRPD pattern, a DSC graph, a TGA graph, or a MSA graph, includes a pattern or graph that is not necessarily identical to those depicted herein, but falls within the limits of experimental errors or deviations when considered by one of ordinary skill in the art.

As used herein, the term “substantially free of” means that the composition contains the indicated substance or substances in an amount of less than about 25%, less than about 20%, less than about 15%, less than about 10%, less than about 5%, less than about 4%, less than about 3%, less than about 2%, or less than about 1% by weight.

### Polymorphs

In one aspect, provided herein is a polymorph of Compound I, which has the structure shown below. In some embodiments, the polymorph is solvated. In some embodiments, the polymorph is not solvated.

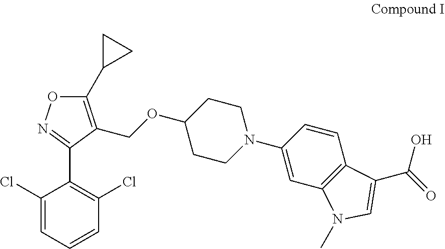 [SMILES: Cn1cc(C(=O)O)c2ccc(N3CCC(OCc4c(-c5c(Cl)cccc5Cl)noc4C4CC4)CC3)cc21]

The polymorphs may have properties such as bioavailability and stability under certain conditions that are suitable for medical or pharmaceutical uses.

A polymorph of Compound I may provide the advantages of bioavailability and stability and may be suitable for use as an active agent in a pharmaceutical composition. Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, polymorphs may provide desired or suitable hygroscopicity, particle size control, dissolution rate, solubility, purity, physical and chemical stability, manufacturability, yield, reproducibility, and/or process control. Thus, polymorphs of Compound I may provide advantages of improving the manufacturing process of an active agent or the stability or storability of a drug product form of the active agent, or having suitable bioavailability and/or stability as an active agent.

The use of certain conditions, such as the use of different solvents and/or temperatures, has been found to produce different polymorphs of Compound I, including polymorphic Forms I-VII described herein, which may exhibit one or more favorable characteristics described herein. The processes for the preparation of the polymorphs described herein and characterization of these polymorphs are described in greater detail below.

### Form I

In some embodiments, provided herein is polymorphic Form I of Compound I.

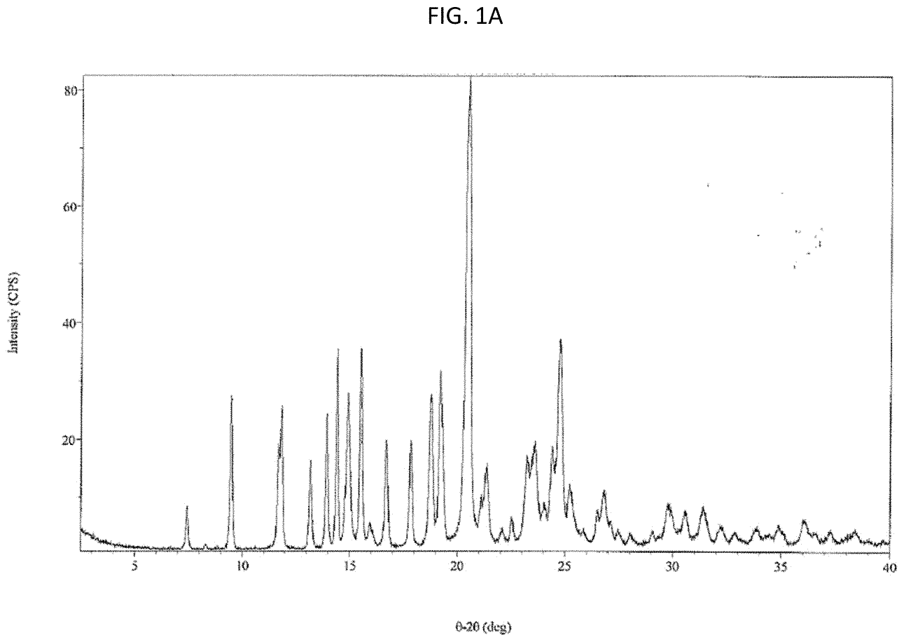

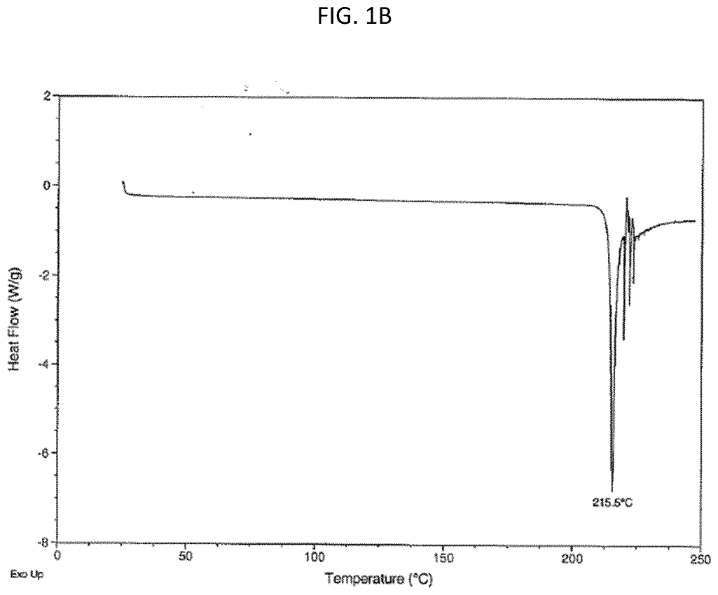

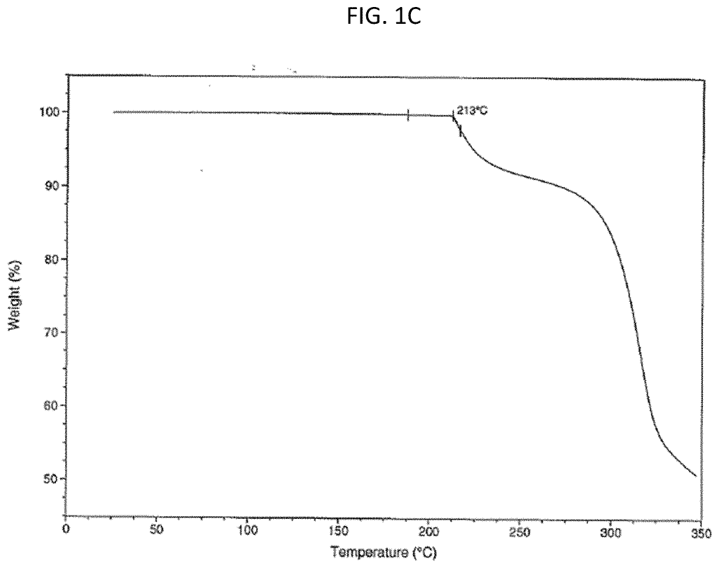

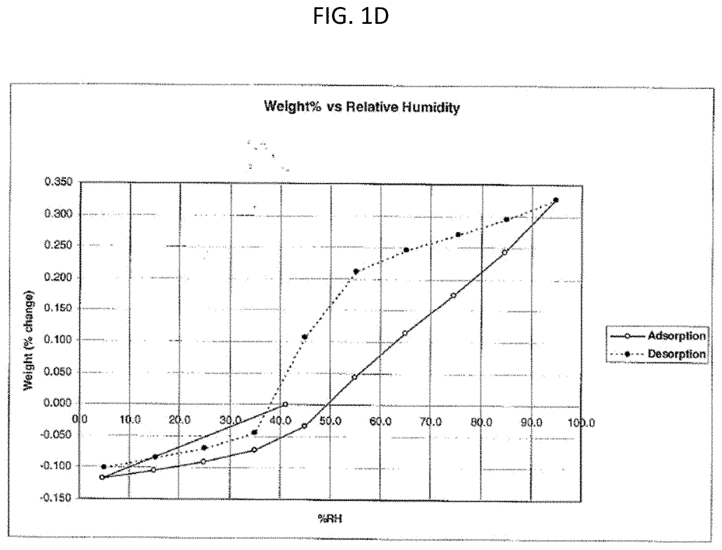

In some embodiments, Form I has an XRPD pattern substantially as shown in FIG. 1A. Angles 2-theta and relative peak intensities that may be observed for Form I using XRPD are shown in Table 1.

TABLE 1

| Angle/2θ | Intensity % |
|---|---|
| 7.44 | 10 |
| 8.30 | 2 |
| 9.48 | 34 |
| 11.69 | 24 |
| 11.81 | 32 |
| 13.16 | 19 |
| 13.92 | 30 |
| 14.40 | 43 |
| 14.92 | 35 |
| 15.51 | 43 |
| 15.94 | 7 |
| 16.70 | 24 |
| 17.84 | 25 |
| 18.77 | 33 |
| 19.20 | 39 |
| 20.48 | 100 |
| 21.12 | 13 |
| 21.35 | 19 |
| 22.08 | 6 |
| 22.54 | 8 |
| 23.24 | 20 |
| 23.60 | 24 |
| 24.04 | 11 |
| 24.41 | 23 |
| 24.74 | 45 |
| 25.23 | 14 |
| 26.51 | 9 |
| 26.81 | 14 |
| 27.13 | 7 |
| 27.47 | 5 |
| 28.03 | 5 |
| 29.05 | 5 |
| 29.78 | 10 |
| 30.57 | 10 |
| 31.38 | 10 |

In some embodiments, polymorphic Form I has an XRPD pattern displaying at least two, at least three, at least four, at least five, at least six, at least seven, at least eight, at least nine, or at least ten of the peaks at angles 2-theta with the greatest intensity in the XRPD pattern as shown in FIG. 1A or as provided in Table 1. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form I, can vary by ±0.6 degrees, ±0.4 degrees, ±0.2 degrees, or ±0.1 degrees 2-theta. In some embodiments, peak assignments listed herein, including for polymorphic Form I, can vary by ±0.6 degrees 2-theta. In some embodiments, peak assignments listed herein, including for polymorphic Form I, can vary by ±0.4 degrees 2-theta. In some embodiments, peak assignments listed herein, including for polymorphic Form I, can vary by ±0.2 degrees 2-theta. In some embodiments, peak assignments listed herein, including for polymorphic Form I, can vary by ±0.1 degrees 2-theta.

In some embodiments, polymorphic Form I has an XRPD pattern comprising peaks at angles 2-theta of 14.40±0.20, 20.48±0.20, and 24.74±0.20 degrees. In some embodiments, polymorphic Form I has an XRPD pattern comprising peaks at angles 2-theta of 14.40±0.20, 15.51±0.20, 19.20±0.20, 20.48±0.20, and 24.74±0.20 degrees. In some embodiments, polymorphic Form I has an XRPD pattern comprising peaks at angles 2-theta of 9.48±0.20, 11.81±0.20, 13.92±0.20, 14.40±0.20, 14.92±0.20, 15.51±0.20, 18.77±0.20, 19.20±0.20, 20.48±0.20, and 24.74±0.20 degrees.

In some embodiments, Form I has a DSC graph substantially as shown in FIG. 1B. In some embodiments, Form I is characterized as having an endotherm onset at about 215.5° C. as determined by DSC. In some embodiments, Form I is characterized as having an endotherm onset at 215.5±2° C. (e.g., 215.5±1.9° C., 215.5±1.8° C., 215.5±1.7° C., 215.5±1.6° C., 215.5±1.5° C., 215.5±1.4° C., 215.5±1.3° C., 215.5±1.2° C., 215.5±1° C., 215.5±0.9° C., 215.5±0.8° C., 215.5±0.7° C., 215.5±0.6° C., 215.5±0.5° C., 215.5±0.4° C., 215.5±0.3° C., 215.5±0.2° C., or 215.5±0.1° C.) as determined by DSC.

In some embodiments, Form I has a TGA graph substantially as shown in FIG. 1C. In some embodiments, Form I shows no weight loss below about 213.0° C. as determined by TGA.

In some embodiments, Form I has a MSA graph substantially as shown in FIG. 1D.

In some embodiments of Form I, at least one, at least two, at least three, at least four, at least five, at least six, or all of the following (a)-(g) apply:

(a) Form I has an XRPD pattern comprising peaks at angles 2-theta of 14.40±0.20, 20.48±0.20, and 24.74±0.20 degrees; an XRPD pattern comprising peaks at angles 2-theta of 14.40±0.20, 15.51±0.20, 19.20±0.20, 20.48±0.20, and 24.74±0.20 degrees; or an XRPD pattern comprising peaks at angles 2-theta of 9.48±0.20, 11.81±0.20, 13.92±0.20, 14.40±0.20, 14.92±0.20, 15.51±0.20, 18.77±0.20, 19.20±0.20, 20.48±0.20, and 24.74±0.20 degrees;
(b) Form I has an XRPD pattern substantially as shown in FIG. 1A;
(c) Form I is characterized as having an endotherm onset at about 215.5° C. as determined by DSC;
(d) Form I has a DSC graph substantially as shown in FIG. 1B;
(e) Form I shows no weight loss below about 213.0° C. as determined by TGA;
(f) Form I has a TGA graph substantially as shown in FIG. 1C; and
(g) Form I has a MSA graph substantially as shown in FIG. 1D.

### Form II

In some embodiments, provided herein is polymorphic Form II of Compound I.

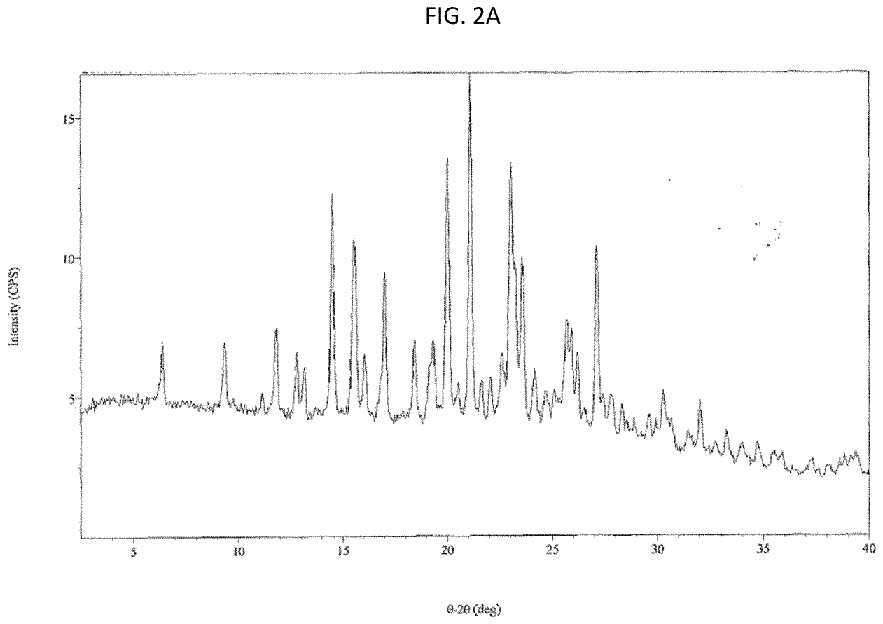

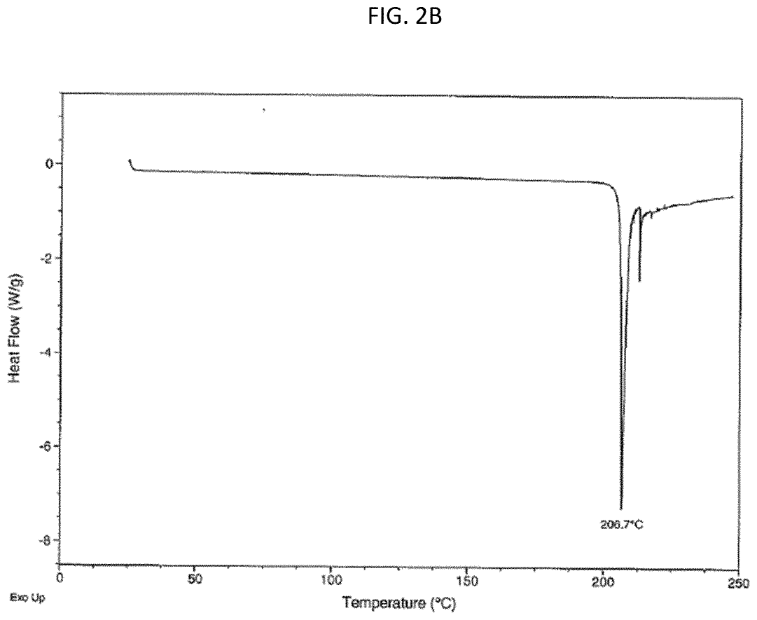

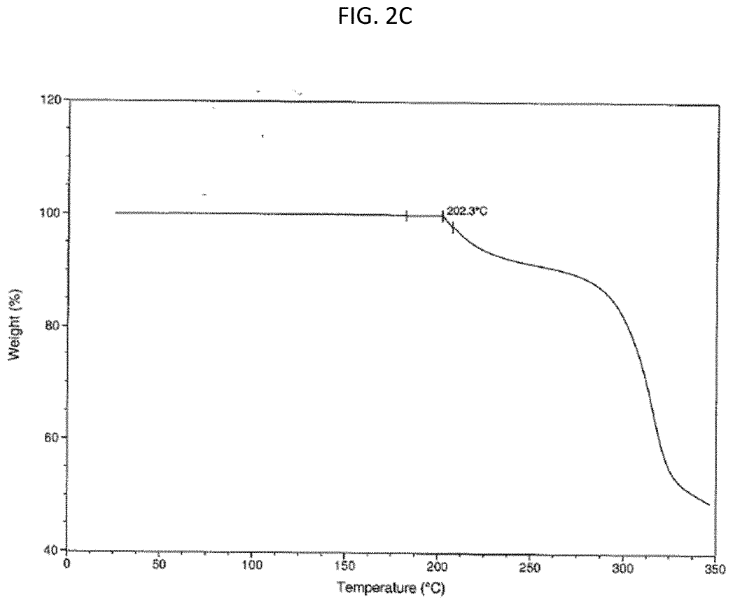

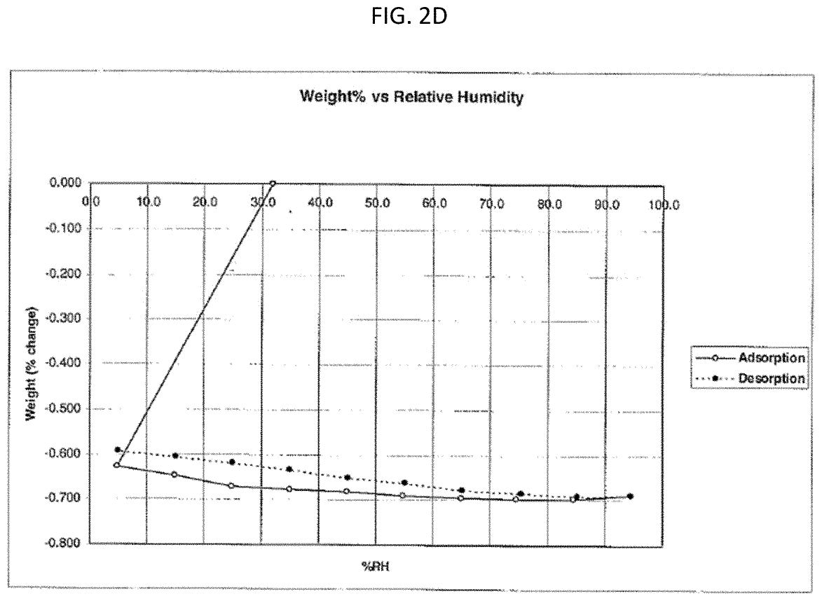

In some embodiments, Form II has an XRPD pattern substantially as shown in FIG. 2A. Angles 2-theta and relative peak intensities that may be observed for Form II using XRPD are shown in Table 2.

TABLE 2

| Angle/2θ | Intensity % |
|---|---|
| 6.39 | 42 |
| 9.37 | 42 |
| 11.18 | 31 |
| 11.82 | 45 |
| 12.80 | 39 |
| 13.16 | 37 |
| 14.50 | 73 |
| 15.56 | 64 |
| 16.05 | 39 |
| 17.01 | 57 |
| 18.43 | 42 |
| 19.13 | 37 |
| 19.33 | 42 |
| 20.00 | 81 |
| 20.52 | 33 |
| 21.09 | 100 |
| 21.62 | 33 |
| 22.05 | 34 |
| 22.61 | 39 |
| 23.04 | 81 |
| 23.24 | 59 |
| 23.58 | 60 |
| 24.15 | 36 |
| 24.68 | 31 |
| 25.09 | 31 |
| 25.69 | 47 |
| 25.91 | 44 |
| 26.20 | 39 |
| 26.52 | 28 |
| 27.13 | 61 |
| 27.39 | 31 |
| 27.79 | 30 |
| 28.31 | 28 |
| 28.89 | 26 |
| 29.63 | 26 |
| 30.27 | 31 |

In some embodiments, polymorphic Form II has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 2A or as provided in Table 2. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form II, can vary by ±0.6 degrees, ±0.4 degrees, ±0.2 degrees, or ±0.1 degrees 2-theta. In some embodiments, peak assignments listed herein, including for polymorphic Form II, can vary by ±0.6 degrees 2-theta. In some embodiments, peak assignments listed herein, including for polymorphic Form II, can vary by ±0.4 degrees 2-theta. In some embodiments, peak assignments listed herein, including for polymorphic Form II, can vary by ±0.2 degrees 2-theta. In some embodiments, peak assignments listed herein, including for polymorphic Form II, can vary by ±0.1 degrees 2-theta.

In some embodiments, polymorphic Form II has an XRPD pattern comprising peaks at angles 2-theta of 20.00±0.20, 21.09±0.20, and 23.04±0.20 degrees. In some embodiments, polymorphic Form II has an XRPD pattern comprising peaks at angles 2-theta of 14.50±0.20, 15.56±0.20, 20.00±0.20, 21.09±0.20, and 23.04±0.20 degrees. In some embodiments, polymorphic Form II has an XRPD pattern comprising peaks at angles 2-theta of 14.50±0.20, 15.56±0.20, 17.01±0.20, 20.00±0.20, 21.09±0.20, 23.04±0.20, 23.24±0.20, 23.58±0.20, 25.69±0.20, and 27.13±0.20 degrees.

In some embodiments, Form II has a DSC graph substantially as shown in FIG. 2B. In some embodiments, Form II is characterized as having an endotherm onset at about 206.7° C. as determined by DSC. In some embodiments, Form II is characterized as having an endotherm onset at about 206.7±2° C. (e.g., 206.7±1.9° C., 206.7±1.8° C., 206.7±1.7° C., 206.7±1.6° C., 206.7±1.5° C., 206.7±1.4° C., 206.7±1.3° C., 206.7±1.2° C., 206.7±1.1° C., 206.7±1° C., 206.7±0.9° C., 206.7±0.8° C., 206.7±0.7° C., 206.7±0.6° C., 206.7±0.5° C., 206.7±0.4° C., 206.7±0.3° C., 206.7±0.2° C., or 206.7±0.1° C.) as determined by DSC.

In some embodiments, Form II has a TGA graph substantially as shown in FIG. 2C. In some embodiments, Form II shows no weight loss below about 202.3° C. as determined by TGA.

In some embodiments, Form II has a MSA graph substantially as shown in FIG. 2D.

In some embodiments of Form II, at least one, at least two, at least three, at least four, at least five, at least six, or all of the following (a)-(g) apply:

(a) Form II has an XRPD pattern comprising peaks at angles 2-theta of 20.00±0.20, 21.09±0.20, and 23.04±0.20 degrees; an XRPD pattern comprising peaks at angles 2-theta of 14.50±0.20, 15.56±0.20, 20.00±0.20, 21.09±0.20, and 23.04±0.20 degrees; or an XRPD pattern comprising peaks at angles 2-theta of 14.50±0.20, 15.56±0.20, 17.01±0.20, 20.00±0.20, 21.09±0.20, 23.04±0.20, 23.24±0.20, 23.58±0.20, 25.69±0.20, and 27.13±0.20 degrees;
(b) Form II has an XRPD pattern substantially as shown in FIG. 2A;
(c) Form II is characterized as having an endotherm onset at about 206.7° C. as determined by DSC;
(d) Form II has a DSC graph substantially as shown in FIG. 2B;
(e) Form II shows no weight loss below about 202.3° C. as determined by TGA;
(f) Form II has a TGA graph substantially as shown in FIG. 2C; and
(g) Form II has a MSA graph substantially as shown in FIG. 2D.

### Form III

In some embodiments, provided herein is polymorphic Form III of Compound I.

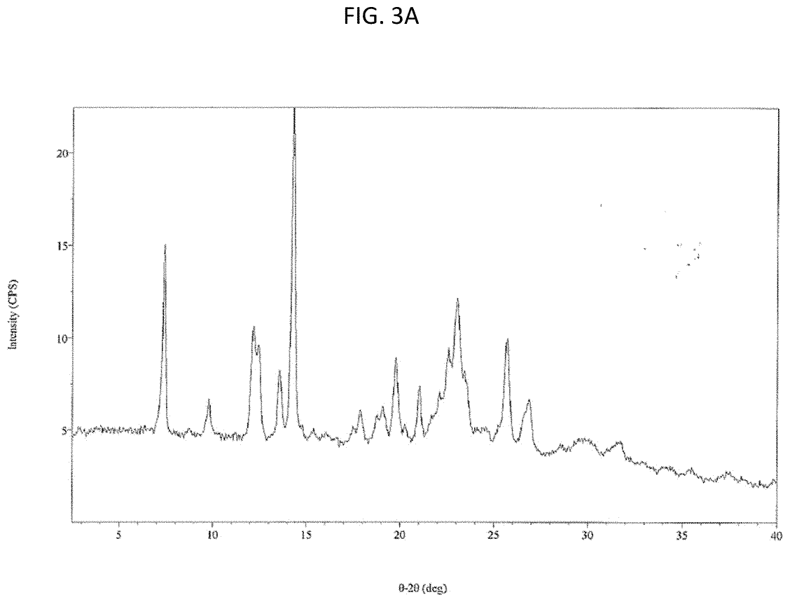

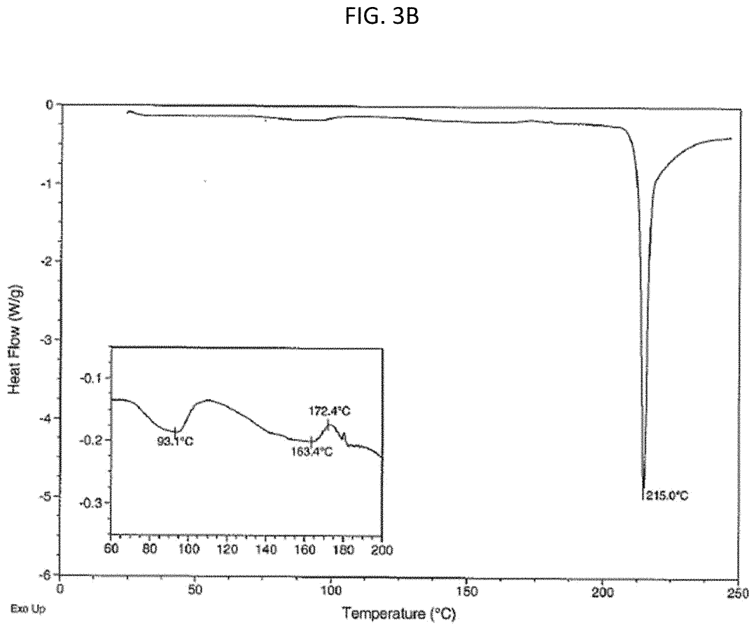

In some embodiments, Form III has an XRPD pattern substantially as shown in FIG. 3A. Angles 2-theta and relative peak intensities that may be observed for Form III using XRPD are shown in Table 3.

TABLE 3

| Angle/2θ | Intensity % |
|---|---|
| 7.40 | 67 |
| 9.79 | 30 |
| 12.16 | 48 |
| 12.43 | 43 |
| 13.56 | 37 |
| 14.27 | 100 |
| 15.39 | 23 |
| 16.03 | 22 |
| 17.50 | 23 |
| 17.87 | 27 |
| 18.75 | 26 |
| 19.06 | 28 |
| 19.77 | 40 |
| 20.25 | 24 |
| 21.03 | 33 |
| 22.10 | 32 |
| 22.58 | 42 |
| 23.04 | 55 |
| 25.69 | 44 |
| 26.85 | 30 |

In some embodiments, polymorphic Form III has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 3A or as provided in Table 3. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form III, can vary by ±0.6 degrees, ±0.4 degrees, ±0.2 degrees, or ±0.1 degrees 2-theta. In some embodiments, peak assignments listed herein, including for polymorphic Form III, can vary by ±0.6 degrees 2-theta. In some embodiments, peak assignments listed herein, including for polymorphic Form III, can vary by ±0.4 degrees 2-theta. In some embodiments, peak assignments listed herein, including for polymorphic Form III, can vary by ±0.2 degrees 2-theta. In some embodiments, peak assignments listed herein, including for polymorphic Form III, can vary by ±0.1 degrees 2-theta.

In some embodiments, polymorphic Form III has an XRPD pattern comprising peaks at angles 2-theta of 7.40±0.20, 14.27±0.20, and 23.04±0.20 degrees. In some embodiments, polymorphic Form III has an XRPD pattern comprising peaks at angles 2-theta of 7.40±0.20, 12.16±0.20, 14.27±0.20, 23.04±0.20, and 25.69±0.20 degrees. In some embodiments, polymorphic Form III has an XRPD pattern comprising peaks at angles 2-theta of 7.40±0.20, 12.16±0.20, 12.43±0.20, 13.56±0.20, 14.27±0.20, 19.77±0.20, 21.03±0.20, 22.58±0.20, 23.04±0.20, and 25.69±0.20 degrees.

In some embodiments, Form III has a DSC graph substantially as shown in FIG. 3B. In some embodiments, Form III is characterized as having an endotherm onset at about 215.0° C. as determined by DSC. In some embodiments, Form III is characterized as having an endotherm onset at about 215.0±2° C. (e.g., 215.0±1.9° C., 215.0±1.8° C., 215.0±1.7° C., 215.0±1.6° C., 215.0±1.5° C., 215.0±1.4° C., 215.0±1.3° C., 215.0±1.2° C., 215.0±1.1° C., 215.0±1° C., 215.0±0.9° C., 215.0±0.8° C., 215.0±0.7° C., 215.0±0.6° C., 215.0±0.5° C., 215.0±0.4° C., 215.0±0.3° C., 215.0±0.2° C., or 215.0±0.1° C.) as determined by DSC.

In some embodiments of Form III, at least one, at least two, at least three, all of the following (a)-(d) apply:

(a) Form III has an XRPD pattern comprising peaks at angles 2-theta of 7.40±0.20, 14.27±0.20, and 23.04±0.20 degrees; an XRPD pattern comprising peaks at angles 2-theta of 7.40±0.20, 12.16±0.20, 14.27±0.20, 23.04±0.20, and 25.69±0.20 degrees; or an XRPD pattern comprising peaks at angles 2-theta of 7.40±0.20, 12.16±0.20, 12.43±0.20, 13.56±0.20, 14.27±0.20, 19.77±0.20, 21.03±0.20, 22.58±0.20, 23.04±0.20, and 25.69±0.20 degrees;
(b) Form III has an XRPD pattern substantially as shown in FIG. 3A;
(c) Form III is characterized as having an endotherm onset at about 215.0° C. as determined by DSC; and
(d) Form III has a DSC graph substantially as shown in FIG. 3B.

### Form IV

In some embodiments, provided herein is polymorphic Form IV of Compound I.

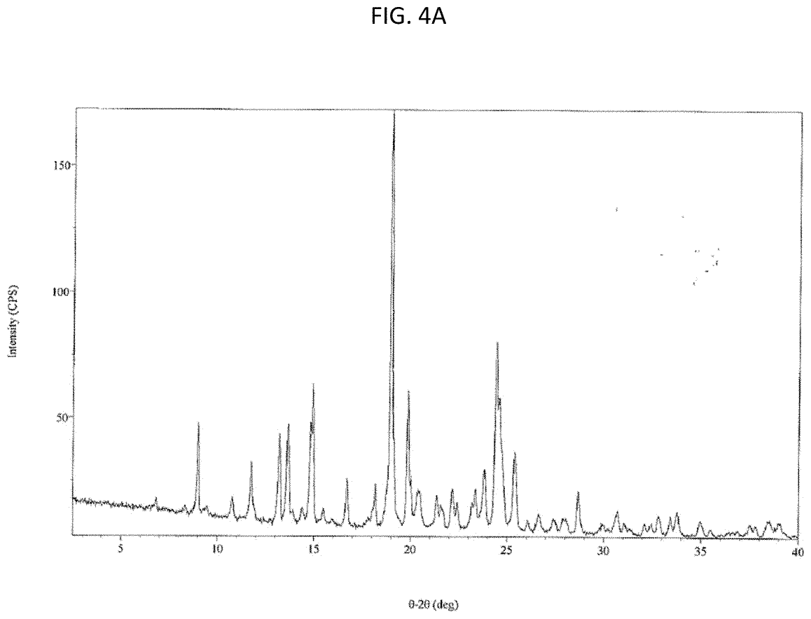

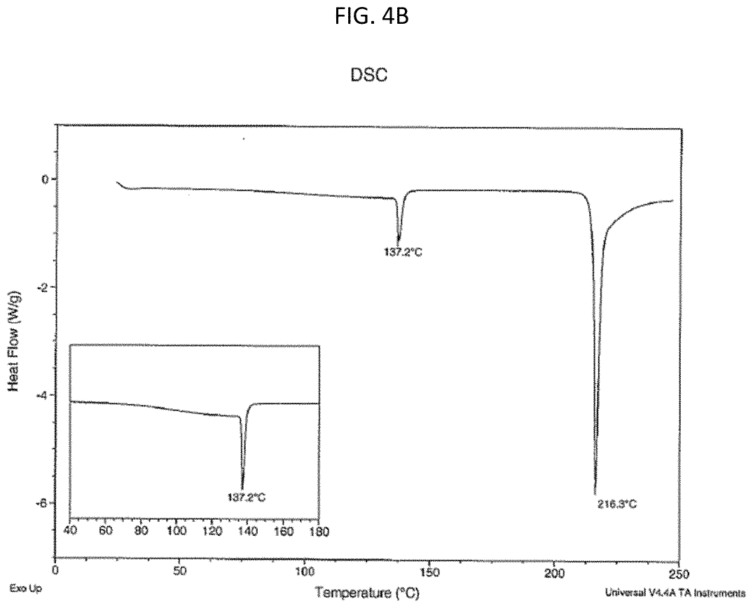

In some embodiments, Form IV has an XRPD pattern substantially as shown in FIG. 4A. Angles 2-theta and relative peak intensities that may be observed for Form IV using XRPD are shown in Table 4.

TABLE 4

| Angle/2θ | Intensity % |
|---|---|
| 6.81 | 10 |
| 8.99 | 27 |
| 10.77 | 11 |
| 11.75 | 19 |
| 13.20 | 25 |
| 13.64 | 27 |
| 14.83 | 28 |
| 14.93 | 37 |
| 16.71 | 15 |
| 18.18 | 14 |
| 18.97 | 100 |
| 19.86 | 35 |
| 20.02 | 15 |
| 21.36 | 11 |
| 21.59 | 9 |
| 22.17 | 12 |
| 22.43 | 10 |
| 23.20 | 10 |
| 23.37 | 13 |
| 23.82 | 17 |
| 24.43 | 47 |
| 24.58 | 33 |
| 25.40 | 21 |
| 26.08 | 6 |
| 26.65 | 7 |
| 27.40 | 6 |
| 27.91 | 6 |
| 28.04 | 6 |
| 28.69 | 12 |

In some embodiments, polymorphic Form IV has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 4A or as provided in Table 4. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form IV, can vary by ±0.6 degrees, ±0.4 degrees, ±0.2 degrees, or ±0.1 degrees 2-theta. In some embodiments, peak assignments listed herein, including for polymorphic Form IV, can vary by ±0.6 degrees 2-theta. In some embodiments, peak assignments listed herein, including for polymorphic Form IV, can vary by ±0.4 degrees 2-theta. In some embodiments, peak assignments listed herein, including for polymorphic Form IV, can vary by ±0.2 degrees 2-theta. In some embodiments, peak assignments listed herein, including for polymorphic Form IV, can vary by ±0.1 degrees 2-theta.

In some embodiments, polymorphic Form IV has an XRPD pattern comprising peaks at angles 2-theta of 14.93±0.20, 18.97±0.20, and 24.43±0.20 degrees. In some embodiments, polymorphic Form IV has an XRPD pattern comprising peaks at angles 2-theta of 14.93±0.20, 18.97±0.20, 19.86±0.20, 24.43±0.20, and 24.58±0.20 degrees. In some embodiments, polymorphic Form IV has an XRPD pattern comprising peaks at angles 2-theta of 8.99±0.20, 13.20±0.20, 13.64±0.20, 14.83±0.20, 14.93±0.20, 18.97±0.20, 19.86±0.20, 24.43±0.20, 24.58±0.20, and 25.40±0.20 degrees.

In some embodiments, Form IV has a DSC graph substantially as shown in FIG. 4B. In some embodiments, Form IV is characterized as having an endotherm onset at about 216.3° C. as determined by DSC. In some embodiments, Form IV is characterized as having an endotherm onset at about 216.3±2° C. (e.g., 216.3±1.9° C., 216.3±1.8° C., 216.3±1.7° C., 216.3±1.6° C., 216.3±1.5° C., 216.3±1.4° C., 216.3±1.3° C., 216.3±1.2° C., 216.3±1.1° C., 216.3±1° C., 216.3±0.9° C., 216.3±0.8° C., 216.3±0.7° C., 216.3±0.6° C., 216.3±0.5° C., 216.3±0.4° C., 216.3±0.3° C., 216.3±0.2° C., or 216.3±0.1° C.) as determined by DSC.

In some embodiments of Form IV, at least one, at least two, at least three, all of the following (a)-(d) apply:

(a) Form IV has an XRPD pattern comprising peaks at angles 2-theta of 14.93±0.20, 18.97±0.20, and 24.43±0.20 degrees; an XRPD pattern comprising peaks at angles 2-theta of 14.93±0.20, 18.97±0.20, 19.86±0.20, 24.43±0.20, and 24.58±0.20 degrees; or an XRPD pattern comprising peaks at angles 2-theta of 8.99±0.20, 13.20±0.20, 13.64±0.20, 14.83±0.20, 14.93±0.20, 18.97±0.20, 19.86±0.20, 24.43±0.20, 24.58±0.20, and 25.40±0.20 degrees;
(b) Form IV has an XRPD pattern substantially as shown in FIG. 4A;
(c) Form IV is characterized as having an endotherm onset at about 216.3° C. as determined by DSC; and
(d) Form IV has a DSC graph substantially as shown in FIG. 4B.

### Form V

In some embodiments, provided herein is polymorphic Form V of Compound I.

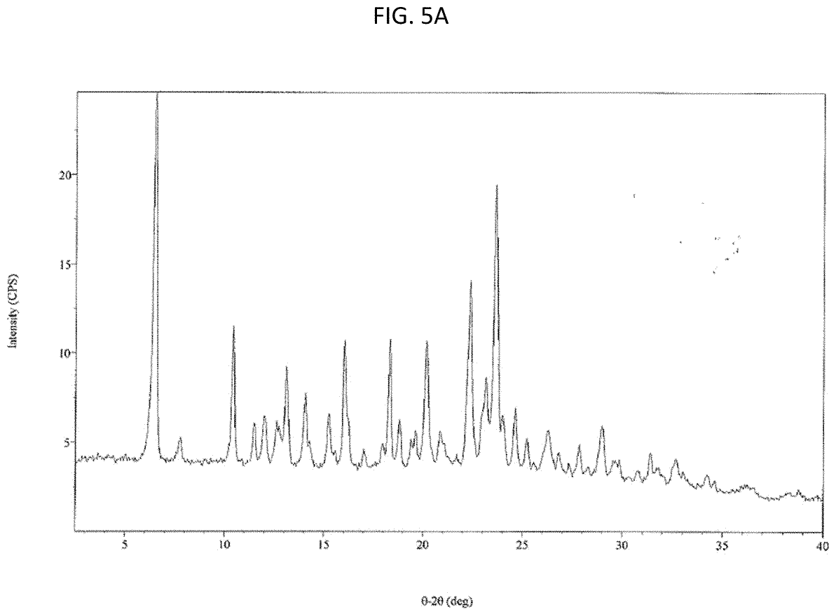

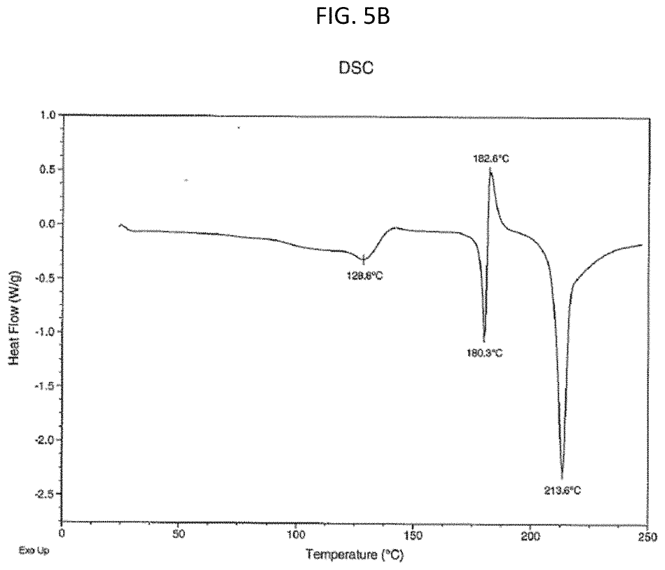

In some embodiments, Form V has an XRPD pattern substantially as shown in FIG. 5A. Angles 2-theta and relative peak intensities that may be observed for Form V using XRPD are shown in Table 5.

TABLE 5

| Angle/2θ | Intensity % |
|---|---|
| 6.49 | 100 |
| 7.77 | 22 |
| 10.44 | 48 |
| 11.49 | 25 |
| 12.02 | 26 |
| 12.62 | 25 |
| 12.74 | 24 |
| 13.1 | 38 |
| 14.06 | 31 |
| 14.27 | 21 |
| 15.25 | 27 |
| 15.56 | 19 |
| 16.04 | 44 |
| 17.01 | 19 |
| 17.96 | 20 |
| 18.31 | 44 |
| 18.8 | 26 |
| 19.39 | 21 |
| 19.62 | 23 |
| 20.16 | 44 |
| 20.85 | 23 |
| 21.68 | 18 |
| 22.36 | 57 |
| 23.15 | 35 |
| 23.63 | 80 |
| 23.99 | 27 |
| 24.62 | 28 |
| 25.21 | 21 |
| 25.55 | 16 |
| 26.26 | 23 |
| 26.8 | 18 |
| 27.32 | 16 |
| 27.84 | 20 |
| 28.28 | 15 |
| 28.97 | 24 |

In some embodiments, polymorphic Form V has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 5A or as provided in Table 5. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form V, can vary by ±0.6 degrees, ±0.4 degrees, ±0.2 degrees, or ±0.1 degrees 2-theta. In some embodiments, peak assignments listed herein, including for polymorphic Form V, can vary by ±0.6 degrees 2-theta. In some embodiments, peak assignments listed herein, including for polymorphic Form V, can vary by ±0.4 degrees 2-theta. In some embodiments, peak assignments listed herein, including for polymorphic Form V, can vary by ±0.2 degrees 2-theta. In some embodiments, peak assignments listed herein, including for polymorphic Form V, can vary by ±0.1 degrees 2-theta.

In some embodiments, polymorphic Form V has an XRPD pattern comprising peaks at angles 2-theta of 6.49±0.20, 22.36±0.20, and 23.63±0.20 degrees. In some embodiments, polymorphic Form V has an XRPD pattern comprising peaks at angles 2-theta of 6.49±0.20, 10.44±0.20, 16.04±0.20, 22.36±0.20, and 23.63±0.20 degrees. In some embodiments, polymorphic Form V has an XRPD pattern comprising peaks at angles 2-theta of 6.49±0.20, 10.44±0.20, 13.10±0.20, 14.06±0.20, 16.04±0.20, 18.31±0.20, 20.16±0.20, 22.36±0.20, 23.15±0.20, and 23.63±0.20 degrees.

In some embodiments, Form V has a DSC graph substantially as shown in FIG. 5B. In some embodiments, Form V is characterized as having an endotherm onset at about 180.3° C., an exotherm onset at about 182.6° C., and/or an endotherm onset at about 213.6° C. as determined by DSC.

In some embodiments of Form V, at least one, at least two, at least three, all of the following (a)-(d) apply:

(a) Form V has an XRPD pattern comprising peaks at angles 2-theta of 6.49±0.20, 22.36±0.20, and 23.63±0.20 degrees; an XRPD pattern comprising peaks at angles 2-theta of 6.49±0.20, 10.44±0.20, 16.04±0.20, 22.36±0.20, and 23.63±0.20 degrees; or an XRPD pattern comprising peaks at angles 2-theta of 6.49±0.20, 10.44±0.20, 13.10±0.20, 14.06±0.20, 16.04±0.20, 18.31±0.20, 20.16±0.20, 22.36±0.20, 23.15±0.20, and 23.63±0.20 degrees;
(b) Form V has an XRPD pattern substantially as shown in FIG. 5A;
(c) Form V is characterized as having an endotherm onset at about 180.3° C., an exotherm onset at about 182.6° C., and/or an endotherm onset at about 213.6° C. as determined by DSC; and
(d) Form V has a DSC graph substantially as shown in FIG. 5B.

### Form VI

In some embodiments, provided herein is polymorphic Form VI of Compound I.

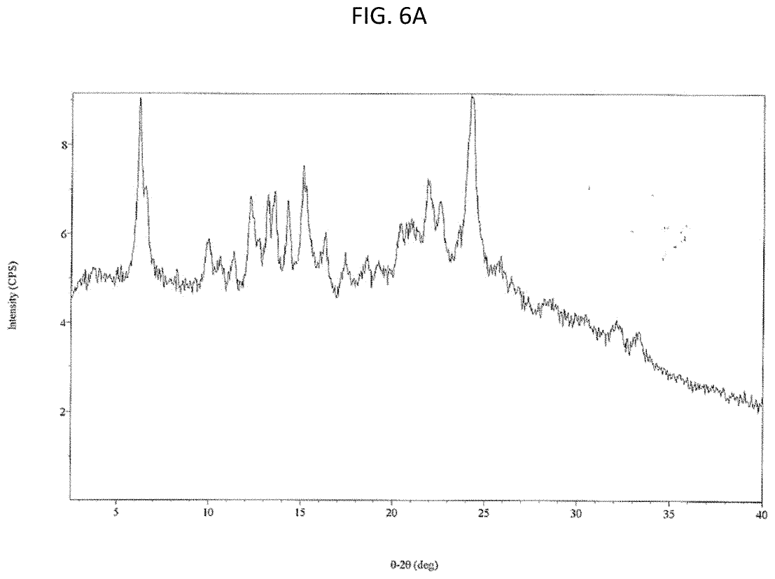

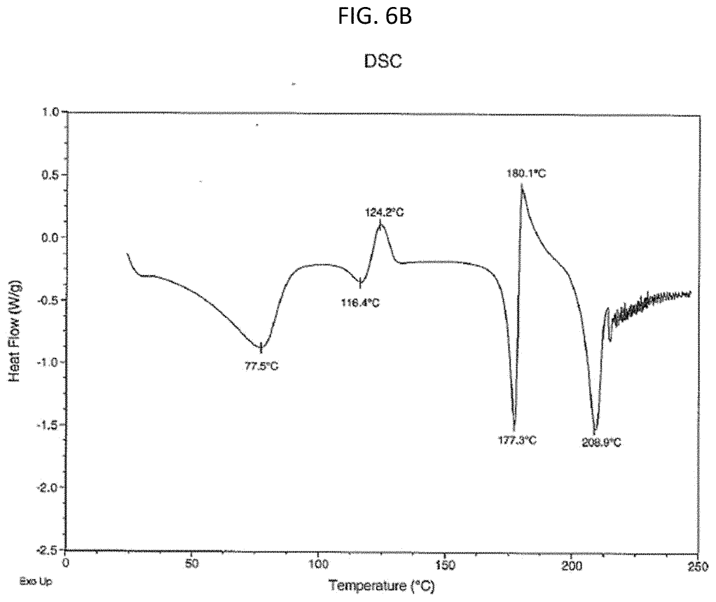

In some embodiments, Form VI has an XRPD pattern substantially as shown in FIG. 6A. Angles 2-theta and relative peak intensities that may be observed for Form VI using XRPD are shown in Table 6.

TABLE 6

| Angle/2θ | Intensity % |
|---|---|
| 6.20 | 98 |
| 6.51 | 78 |
| 9.92 | 64 |
| 10.57 | 60 |
| 11.31 | 62 |
| 12.24 | 75 |
| 12.66 | 64 |
| 13.18 | 76 |
| 13.55 | 76 |
| 14.26 | 74 |
| 15.12 | 82 |
| 16.29 | 66 |
| 17.39 | 61 |
| 18.55 | 60 |
| 19.20 | 59 |
| 20.36 | 67 |
| 21.89 | 78 |
| 22.55 | 74 |
| 24.25 | 100 |

In some embodiments, polymorphic Form VI has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 6A or as provided in Table 6. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form VI, can vary by ±0.6 degrees, ±0.4 degrees, ±0.2 degrees, or ±0.1 degrees 2-theta. In some embodiments, peak assignments listed herein, including for polymorphic Form VI, can vary by ±0.6 degrees 2-theta. In some embodiments, peak assignments listed herein, including for polymorphic Form VI, can vary by ±0.4 degrees 2-theta. In some embodiments, peak assignments listed herein, including for polymorphic Form VI, can vary by ±0.2 degrees 2-theta. In some embodiments, peak assignments listed herein, including for polymorphic Form VI, can vary by ±0.1 degrees 2-theta.

In some embodiments, polymorphic Form VI has an XRPD pattern comprising peaks at angles 2-theta of 6.20±0.20, 15.12±0.20, and 24.25±0.20 degrees. In some embodiments, polymorphic Form VI has an XRPD pattern comprising peaks at angles 2-theta of 6.20±0.20, 6.51±0.20, 15.12±0.20, 21.89±0.20, and 24.25±0.20 degrees. In some embodiments, polymorphic Form VI has an XRPD pattern comprising peaks at angles 2-theta of 6.20±0.20, 6.51±0.20, 12.24±0.20, 13.18±0.20, 13.55±0.20, 14.26±0.20, 15.12±0.20, 21.89±0.20, 22.55±0.20, and 24.25±0.20 degrees.

In some embodiments, Form VI has a DSC graph substantially as shown in FIG. 6B. In some embodiments, Form VI is characterized as having an endotherm onset at about 177.3° C., an exotherm onset at about 180.1° C., and/or an endotherm onset at about 208.9° C. as determined by DSC.

In some embodiments of Form VI, at least one, at least two, at least three, all of the following (a)-(d) apply:

(a) Form VI has an XRPD pattern comprising peaks at angles 2-theta of 6.20±0.20, 15.12±0.20, and 24.25±0.20 degrees; an XRPD pattern comprising peaks at angles 2-theta of 6.20±0.20, 6.51±0.20, 15.12±0.20, 21.89±0.20, and 24.25±0.20 degrees; or an XRPD pattern comprising peaks at angles 2-theta of 6.20±0.20, 6.51±0.20, 12.24±0.20, 13.18±0.20, 13.55±0.20, 14.26±0.20, 15.12±0.20, 21.89±0.20, 22.55±0.20, and 24.25±0.20 degrees;
(b) Form VI has an XRPD pattern substantially as shown in FIG. 6A;
(c) Form VI is characterized as having an endotherm onset at about 177.3° C., an exotherm onset at about 180.1° C., and/or an endotherm onset at about 208.9° C. as determined by DSC; and
(d) Form VI has a DSC graph substantially as shown in FIG. 6B.

### Form VII

In some embodiments, provided herein is polymorphic Form VII of Compound I.

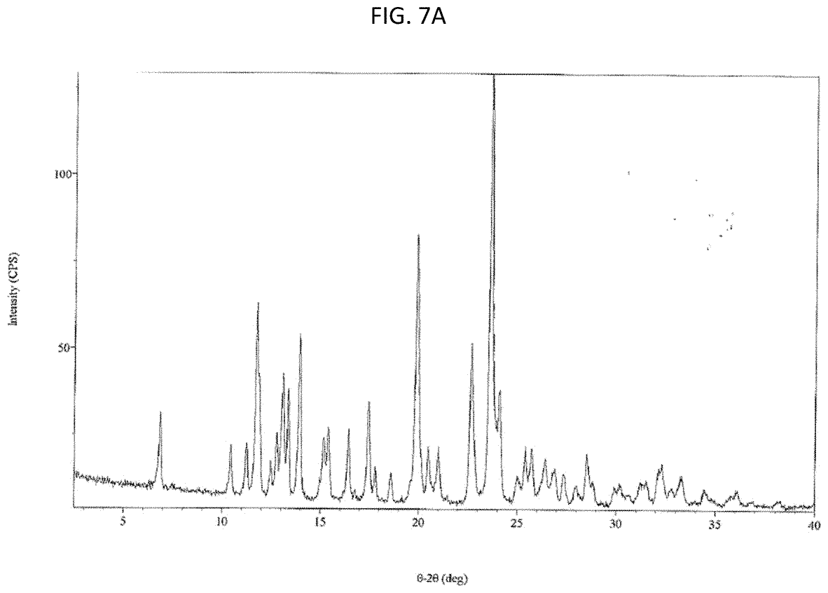

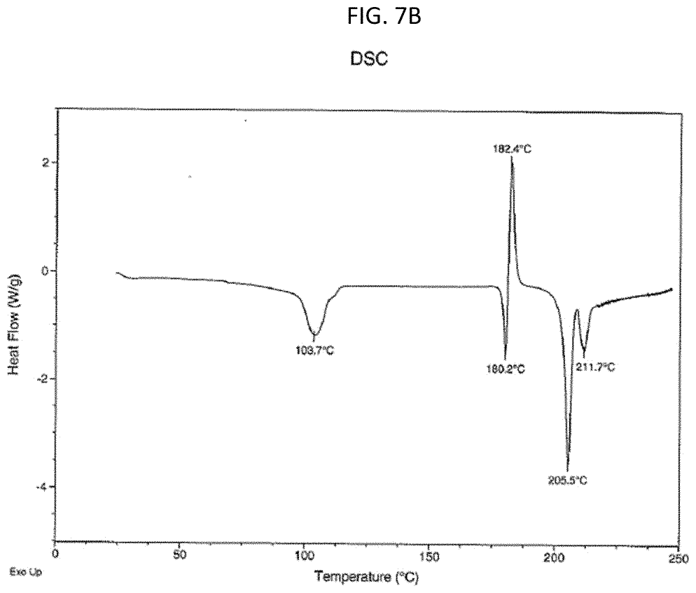

In some embodiments, Form VII has an XRPD pattern substantially as shown in FIG. 7A. Angles 2-theta and relative peak intensities that may be observed for Form VII using XRPD are shown in Table 7.

TABLE 7

| Angle/2θ | Intensity % |
|---|---|
| 6.85 | 24 |
| 10.44 | 17 |
| 11.24 | 17 |
| 11.74 | 49 |
| 11.85 | 33 |
| 12.48 | 14 |
| 12.77 | 20 |
| 13.08 | 33 |
| 13.36 | 30 |
| 13.94 | 42 |
| 15.16 | 19 |
| 15.39 | 21 |
| 16.43 | 21 |
| 17.44 | 27 |
| 17.79 | 12 |
| 18.58 | 11 |
| 19.88 | 64 |
| 20.47 | 17 |
| 20.99 | 17 |
| 22.67 | 38 |
| 23.63 | 100 |
| 24.08 | 29 |
| 25.02 | 11 |
| 25.4 | 16 |
| 25.72 | 17 |
| 26.41 | 14 |
| 26.9 | 12 |
| 27.36 | 11 |
| 27.98 | 8 |
| 28.52 | 15 |
| 28.79 | 9 |

In some embodiments, polymorphic Form VII has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 7A or as provided in Table 7. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form VII, can vary by ±0.6 degrees, ±0.4 degrees, ±0.2 degrees, or ±0.1 degrees 2-theta. In some embodiments, peak assignments listed herein, including for polymorphic Form VII, can vary by ±0.6 degrees 2-theta. In some embodiments, peak assignments listed herein, including for polymorphic Form VII, can vary by ±0.4 degrees 2-theta. In some embodiments, peak assignments listed herein, including for polymorphic Form VII, can vary by ±0.2 degrees 2-theta. In some embodiments, peak assignments listed herein, including for polymorphic Form VII, can vary by ±0.1 degrees 2-theta.

In some embodiments, polymorphic Form VII has an XRPD pattern comprising peaks at angles 2-theta of 11.74±0.20, 19.88±0.20, and 23.63±0.20 degrees. In some embodiments, polymorphic Form VII has an XRPD pattern comprising peaks at angles 2-theta of 11.74±0.20, 13.94±0.20, 19.88±0.20, 22.67±0.20, and 23.63±0.20 degrees. In some embodiments, polymorphic Form VII has an XRPD pattern comprising peaks at angles 2-theta of 11.74±0.20, 11.85±0.20, 13.08±0.20, 13.36±0.20, 13.94±0.20, 17.44±0.20, 19.88±0.20, 22.67±0.20, 23.63±0.20, and 24.08±0.20 degrees.

In some embodiments, Form VII has a DSC graph substantially as shown in FIG. 7B. In some embodiments, Form VII is characterized as having an endotherm onset at about 180.2° C., an exotherm onset at about 182.4° C., an endotherm onset at about 205.5° C., and/or an endotherm onset at about 211.7° C. as determined by DSC.

In some embodiments of Form VII, at least one, at least two, at least three, all of the following (a)-(d) apply:

(a) Form VII has an XRPD pattern comprising peaks at angles 2-theta of 11.74±0.20, 19.88±0.20, and 23.63±0.20 degrees; an XRPD pattern comprising peaks at angles 2-theta of 11.74±0.20, 13.94±0.20, 19.88±0.20, 22.67±0.20, and 23.63±0.20 degrees; or an XRPD pattern comprising peaks at angles 2-theta of 11.74±0.20, 11.85±0.20, 13.08±0.20, 13.36±0.20, 13.94±0.20, 17.44±0.20, 19.88±0.20, 22.67±0.20, 23.63±0.20, and 24.08±0.20 degrees;
(b) Form VII has an XRPD pattern substantially as shown in FIG. 7A;
(c) Form VII is characterized as having an endotherm onset at about 180.2° C., an exotherm onset at about 182.4° C., an endotherm onset at about 205.5° C., and/or an endotherm onset at about 211.7° C. as determined by DSC; and
(d) Form VII has a DSC graph substantially as shown in FIG. 7B.

### Compositions

In another aspect, provided herein is a composition comprising a polymorphic form disclosed herein (e.g., Form I, II, III, IV, V, VI, VII, or a mixture thereof). In some embodiments, the composition comprises Form I. In some embodiments, the composition comprises Form II. In some embodiments, the composition comprises Form III. In some embodiments, the composition comprises Form IV. In some embodiments, the composition comprises Form V. In some embodiments, the composition comprises Form VI. In some embodiments, the composition comprises Form VII. In some embodiments, the composition further comprises a pharmaceutically acceptable carrier.

In some embodiments, provided is a composition comprising Form I of Compound I. In some embodiments, the composition is substantially free of other polymorphic forms of Compound I. In some embodiments, the composition is substantially free of amorphous or non-crystalline form of Compound I.

In some embodiments of the composition comprising Form I of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form I. In some embodiments of the composition comprising Form I of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I exists in Form I.

In some embodiments, provided is a composition comprising Form II of Compound I. In some embodiments, the composition is substantially free of other polymorphic forms of Compound I. In some embodiments, the composition is substantially free of amorphous or non-crystalline form of Compound I.

In some embodiments of the composition comprising Form II of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form II. In some embodiments of the composition comprising Form II of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I exists in Form II.

In some embodiments, provided is a composition comprising Form III of Compound I. In some embodiments, the composition is substantially free of other polymorphic forms of Compound I. In some embodiments, the composition is substantially free of amorphous or non-crystalline form of Compound I.

In some embodiments of the composition comprising Form III of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form III. In some embodiments of the composition comprising Form III of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I exists in Form III.

In some embodiments, provided is a composition comprising Form IV of Compound I. In some embodiments, the composition is substantially free of other polymorphic forms of Compound I. In some embodiments, the composition is substantially free of amorphous or non-crystalline form of Compound I.

In some embodiments of the composition comprising Form IV of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form IV. In some embodiments of the composition comprising Form IV of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I exists in Form IV.

In some embodiments, provided is a composition comprising Form V of Compound I. In some embodiments, the composition is substantially free of other polymorphic forms of Compound I. In some embodiments, the composition is substantially free of amorphous or non-crystalline form of Compound I.

In some embodiments of the composition comprising Form V of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form V. In some embodiments of the composition comprising Form V of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I exists in Form V.

In some embodiments, provided is a composition comprising Form VI of Compound I. In some embodiments, the composition is substantially free of other polymorphic forms of Compound I. In some embodiments, the composition is substantially free of amorphous or non-crystalline form of Compound I.

In some embodiments of the composition comprising Form VI of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form VI. In some embodiments of the composition comprising Form VI of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I exists in Form VI.

In some embodiments, provided is a composition comprising Form VII of Compound I. In some embodiments, the composition is substantially free of other polymorphic forms of Compound I. In some embodiments, the composition is substantially free of amorphous or non-crystalline form of Compound I.

In some embodiments of the composition comprising Form VII of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form VII. In some embodiments of the composition comprising Form VII of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I exists in Form VII.

In some embodiments, provided is a tablet or capsule comprising one or more of the polymorphic forms described herein (e.g., Form I, II, III, IV, V, VI, VII, or a mixture thereof), and one or more pharmaceutically acceptable carriers. In some embodiments, provided is a tablet or capsule comprising substantially pure polymorphic Form I of Compound I, and one or more pharmaceutically acceptable carriers. In some embodiments, provided is a tablet or capsule comprising substantially pure polymorphic Form II of Compound I, and one or more pharmaceutically acceptable carriers. In some embodiments, provided is a tablet or capsule comprising substantially pure polymorphic Form III of Compound I, and one or more pharmaceutically acceptable carriers. In some embodiments, provided is a tablet or capsule comprising substantially pure polymorphic Form IV of Compound I, and one or more pharmaceutically acceptable carriers. In some embodiments, provided is a tablet or capsule comprising substantially pure polymorphic Form V of Compound I, and one or more pharmaceutically acceptable carriers. In some embodiments, provided is a tablet or capsule comprising substantially pure polymorphic Form VI of Compound I, and one or more pharmaceutically acceptable carriers. In some embodiments, provided is a tablet or capsule comprising substantially pure polymorphic Form VII of Compound I, and one or more pharmaceutically acceptable carriers.

### Methods of Preparation

### Form I

In some embodiments, provided is a method of preparing Form I of Compound I, comprising slurrying a solution comprising the compound and a solvent, wherein the solvent comprises an alcohol (e.g., methanol, ethanol, or isopropanol), an acetate (e.g., isopropyl acetate or ethyl acetate), water, or a mixture thereof. In some embodiments, the solvent comprises an alcohol. In some embodiments, the solvent comprises methanol. In some embodiments, the solvent comprises an acetate. In some embodiments, the solvent comprises ethyl acetate. In some embodiments, the solvent comprises a mixture of isopropanol and water. In some embodiment, the slurrying is performed at a temperature of about 25° C.

### Form II

In some embodiments, provided is a method of preparing Form II of Compound I, comprising slurrying a solution comprising the compound and a solvent, wherein the solvent comprises acetone or acetonitrile. In some embodiments, the solvent comprises acetone. In some embodiments, the solvent comprises acetonitrile. In some embodiments, the slurrying is performed at an elevated temperature. In some embodiments, the elevated temperature is about 80° C., about 75° C., about 70° C., about 65° C., about 60° C., about 55° C., about 50° C., about 45° C., or about 40° C.

### Form III

In some embodiments, provided is a method of preparing Form III of Compound I, comprising vapor diffusing a solution comprising the compound and a solvent, wherein the solvent comprises a mixture of tetrahydrofuran (THF) and diethyl ether.

### Form IV

In some embodiments, provided is a method of preparing Form IV of Compound I, comprising slow cooling a solution comprising the compound and a solvent, wherein the solvent comprises a mixture of methanol and water.

### Form V

In some embodiments, provided is a method of preparing Form V of Compound I, comprising vapor diffusing a solution comprising the compound and a solvent, wherein the solvent comprises a mixture of THF and hexane.

### Form VI

In some embodiments, provided is a method of preparing Form VI of Compound I, comprising slow evaporating a solution comprising the compound and a solvent, wherein the solvent comprises a mixture of acetone and acetonitrile.

### Form VII

In some embodiments, provided is a method of preparing Form VII of Compound I, comprising crystalizing a solution comprising the compound and a solvent, wherein the solvent comprises chloroform.

### Methods of Use

In another aspect, provided herein is a method of treating a liver disorder in a patient (e.g., a human patient) in need thereof comprising administering a therapeutically effective amount of a polymorphic form disclosed herein (e.g., Form I, II, III, IV, V, VI, VII, or a mixture thereof). In some embodiments, the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is NAFLD or NASH. In some embodiments, the liver disorder is NAFLD. In some embodiments, the liver disorder is NASH. In some embodiments, the patient has had a liver biopsy. In some embodiments, the method further comprises obtaining the results of a liver biopsy.

In some embodiments, provided is a method of impeding or slowing the progression of NAFLD to NASH in a patient (e.g., a human patient) in need thereof comprising administering a therapeutically effective amount of a polymorphic form disclosed herein (e.g., Form I, II, III, IV, V, VI, VII, or a mixture thereof).

Compound I is preferentially distributed to the liver, which, without being bound by theory, would allow the compound to reach its FXR target in the liver with fewer off-target adverse effects. For example, Compound I has an approximately 20-fold higher concentration in the liver than in the plasma, kidney, lungs, heart, and skin. This trait would likely be particularly beneficial for vulnerable populations, such as children, the elderly, and people with comorbidities.

Further, pruritus is a well-documented adverse effect of several FXR agonists and can result in patient discomfort, a decrease in patient quality of life, and an increased likelihood of ceasing treatment. Pruritus is particularly burdensome for indications, such as those described herein, including NASH, for which chronic drug administration is likely. The tissue specificity of Compound I, in particular the preference for liver over skin tissue is a striking and unpredicted observation that makes it more likely that the compound will not cause pruritus in the skin, a theory that has been substantiated by human trials thus far.

In some embodiments, provided is a method of treating a liver disorder in a patient in need thereof (e.g., a human patient) with an FXR agonist that preferentially distributes in liver tissue over one or more of kidney, lung, heart, and skin tissues, the method comprising administering a therapeutically effective amount of the FXR agonist, wherein the FXR agonist is a polymorphic form disclosed herein (e.g., Form I, II, III, IV, V, VI, VII, or a mixture thereof).

In some embodiments, provided herein is a method of treating a liver disorder in a patient in need thereof with an FXR agonist, such as a polymorphic form disclosed herein (e.g., Form I, II, III, IV, V, VI, VII, or a mixture thereof), wherein the FXR agonist does not activate TGR5 signaling. In some embodiments, the level of an FXR-regulated gene is increased. In some embodiments, the level of small heterodimer partner (SHP), bile salt export pump (BSEP) and fibroblast growth factor 19 (FGF-19) is increased. In some embodiments, the liver disorder is NASH.

In some embodiments, provided herein is a method of reducing liver damage comprising administering an FXR agonist, such as a polymorphic form disclosed herein (e.g., Form I, II, III, IV, V, VI, VII, or a mixture thereof), to an individual in need thereof. In some embodiments, fibrosis is reduced. In some embodiments, the level of expression of one or more markers for fibrosis is reduced. In some embodiments, the level of Ccr2, Col1a1, Col1a2, Col1a3, Cxcr3, Dcn, Hgf, Il1a, Inhbe, Lox, Loxl1, Loxl2, Loxl3, Mmp2, pdgfb, Plau, Serpine1, Perpinh1, Snai, Tgfb1, Tgfb3, Thbs1, Thbs2, Timp2, and/or Timp3 expression is reduced. In some embodiments, the level of collagen is reduced. In some embodiments, the level of collagen fragments is reduced. In some embodiments, the level of expression of the fibrosis marker is reduced at least 2, at least 3, at least 4, or at least 5-fold. In some embodiments, the level of expression of the fibrosis marker is reduced about 2-fold, about 3-fold, about 4-fold, or about 5-fold.

In some embodiments, provided herein a method of reducing liver damage comprising administering an FXR agonist, such as a polymorphic form disclosed herein (e.g., Form I, II, III, IV, V, VI, VII, or a mixture thereof), to an individual in need thereof. In some embodiments, inflammation is reduced. In some embodiments, one or more markers of inflammation are reduced. In some embodiments, the level of expression of Adgre1, Ccr2, Ccr5, II1A, and/or Tlr4 is reduced. In some embodiments, the level of expression of the inflammation marker is reduced at least 2-, at least 3-, at least 4-, or at least 5-fold. In some embodiments, the level of expression of the fibrosis marker is reduced about 2-fold, about 3-fold, about 4-fold, or about 5-fold.

In some embodiments, the administration does not result in pruritus in the patient greater than Grade 2 in severity. In some embodiments, the administration does not result in pruritus in the patient greater than Grade 1 in severity. In some embodiments, the administration does not result in pruritus in the patient. The grading of adverse effects is known. According to Version 5 of the Common Terminology Criteria for Adverse Events (published Nov. 27, 2017), Grade 1 pruritus is characterized as “Mild or localized; topical intervention indicated.” Grade 2 pruritus is characterized as “Widespread and intermittent; skin changes from scratching (e.g., edema, papulation, excoriations, lichenification, oozing/crusts); oral intervention indicated; limiting instrumental ADL.” Grade 3 pruritus is characterized as “Widespread and constant; limiting self care ADL or sleep; systemic corticosteroid or immunosuppressive therapy indicated.” Activities of daily living (ADL) are divided into two categories: “Instrumental ADL refer to preparing meals, shopping for groceries or clothes, using the telephone, managing money, etc.,” and “Self care ADL refer to bathing, dressing and undressing, feeding self, using the toilet, taking medications, and not bedridden.”

Accordingly, in some embodiments, provided herein is a method of treating a liver disorder in a patient (e.g., a human patient) in need thereof with an FXR agonist that does not result in detectable pruritus in the patient, the method comprising administering to the patient in need thereof a therapeutically effective amount of the FXR agonist, wherein the FXR agonist is a polymorphic form disclosed herein (e.g., Form I, II, III, IV, V, VI, VII, or a mixture thereof).

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, or 90 or more.

NAFLD and NASH are common causes of liver transplantation, but patients that already received one liver transplant often develop NAFLD and/or NASH again. Accordingly, in some embodiments, the patient has had a liver transplant.

In some embodiments, the patient's alkaline phosphatase, gamma-glutamyl transferase (GGT), alanine aminotransferase (ALT) and/or aspartate aminotransferase (AST) levels are elevated. In some embodiments, the GGT, ALT, and/or AST levels are elevated prior to treatment with a polymorphic form disclosed herein (e.g., Form I, II, III, IV, V, VI, VII, or a mixture thereof). In some embodiments, the patient's ALT level is about 2-4-fold greater than the upper limit of normal levels. In some embodiments, the patient's AST level is about 2-4-fold greater than the upper limit of normal levels. In some embodiments, the patient's GGT level is about 1.5-3-fold greater than the upper limit of normal levels. In some embodiments, the patient's alkaline phosphatase level is about 1.5-3-fold greater than the upper limit of normal levels. Methods of determining the levels of these molecules are well known. Normal levels of ALT in the blood range from about 7-56 units/liter. Normal levels of AST in the blood range from about 10-40 units/liter. Normal levels of GGT in the blood range from about 9-48 units/liter. Normal levels of alkaline phosphatase in the blood range from about 53-128 units/liter for a 20- to 50-year-old man and about 42-98 units/liter for a 20- to 50-year-old woman.

Accordingly, in some embodiments, a polymorphic form disclosed herein (e.g., Form I, II, III, IV, V, VI, VII, or a mixture thereof), reduces level of AST, ALT, and/or GGT in an individual having elevated AST, ALT, and/or GGT levels. In some embodiments, the level of ALT is reduced at least 2-, at least 3-, at least 4-, or at least 5-fold. In some embodiments, the level of ALT is reduced about 2- to about 5-fold. In some embodiments, the level of AST is reduced at least 2-, at least 3-, at least 4-, or at least 5-fold. In some embodiments, the level of AST is reduced about 1.5 to about 3-fold. In some embodiments, the level of GGT is reduced at least 2, at least 3, at least 4, or at least 5-fold. In some embodiments, the level of GGT is reduced about 1.5 to about 3-fold.

In some embodiments, administration of a polymorphic form disclosed herein (e.g., Form I, II, III, IV, V, VI, VII, or a mixture thereof), to a subject results in a reduced NAFLD Activity Score (NAS). For example, in some embodiments, steatosis, inflammation, and/or ballooning is reduced upon treatment. In some embodiments, the compounds disclosed herein reduce liver fibrosis. In some embodiments, the compounds reduce serum triglycerides. In some embodiments, the compounds reduce liver triglycerides.

In some embodiments, the patient is at risk of developing an adverse effect prior to administering a polymorphic form disclosed herein (e.g., Form I, II, III, IV, V, VI, VII, or a mixture thereof). In some embodiments, the adverse effect is an adverse effect which affects the kidney, lung, heart, and/or skin. In some embodiments, the adverse effect is pruritus.

In some embodiments, the patient has had one or more prior therapies. In some embodiments, the liver disorder progressed during the therapy. In some embodiments, the patient has had one or more prior therapies with another FXR agonist other that Compound I. In some embodiments, the patient suffered from pruritus during at least one of the one or more prior therapies.

In some embodiments, the therapeutically effective amount is below the level that induces an adverse effect in the patient, such as below the level that induces pruritus, such as grade 2 or grade 3 pruritus.

### Methods of Manufacturing a Medicament

In some embodiments, provided is use of a polymorphic form described herein (e.g., Form I, II, III, IV, V, VI, VII, or a mixture thereof) in the manufacture of a medicament for use in a method disclosed herein

### Kits

Also provided are articles of manufacture and kits comprising any of the polymorphic forms or compositions provided herein. The article of manufacture may comprise a container with a label. Suitable containers include, but are not limited to, bottles, vials, and test tubes. The containers may be formed from a variety of materials such as glass or plastic. The container may hold a pharmaceutical composition provided herein. The label on the container may indicate that the pharmaceutical composition is used for treating a condition described herein, and may also indicate directions for either in vivo or in vitro use.

In one aspect, provided herein are kits comprising a polymorphic form or composition described herein and instructions for use. A kit may additionally contain any materials or equipment that may be used in the administration of the polymorphic forms or composition, such as vials, syringes, or IV bags. A kit may also contain sterile packaging.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

The following abbreviations may be used herein:

| | |
|---|---|
| XRPD | X-Ray Powder Diffraction |
| DSC | Differential Scanning Calorimetry |
| TGA | Thermogravimetric Analysis |
| MSA | Moisture Sorption Analysis |
| RH | Relative Humidity |
| RT | Room Temperature |
| ACN | Acetonitrile |
| EtOAc | Ethyl Acetate |
| IPA | Isopropyl Alcohol |
| MeOH | Methanol |
| THF | Tetrahydrofuran |
| SE | Slow Evaporation |
| SC | Slow Cooling |

The polymorphic forms of Compound I were characterized by various analytical techniques, including XRPD, DSC, and TGA, using the procedures described below.

### XRPD

The X-ray powder diffraction (XRPD) analysis was performed using an Inel XRG-3000 diffractometer equipped with a CPS (Curved Position Sensitive) detector with a 2θ range of 120°. Real time data were collected using Cu-Kα radiation at a resolution of 0.03° 2 θ. The tube voltage and current were set to 40 kV and 30 mA, respectively. The monochromator slit was set at 5 mm by 160 μm. The pattern is displayed from 2.5-40° 2 θ. The sample was prepared for analysis by packing it into a thin-walled glass capillary. The capillary was mounted onto a goniometer head that is motorized to permit spinning of the capillary during data acquisition. The sample was analyzed for 5 min Instrument calibration was performed using a silicon reference standard.

### DSC

DSC analyses were performed using a TA Instruments differential scanning calorimeter 2920 or Q2000. Each sample was placed into an aluminum DSC pan, and its weight accurately recorded. The pan was covered with a lid and crimped. The sample cell was equilibrated at 25° C. and heated under a nitrogen purge at a rate of 10° C./min, up to a final temperature of 250° C. Indium metal was used as the calibration standard. Reported temperatures are at the transition maxima.

### TGA

TG analyses were performed using a TA Instruments 2950 thermogravimetric analyzer. Each sample was placed in an aluminum sample pan, inserted into the TG furnace, and accurately weighed. The furnace was first equilibrated at 25° C., and then heated under nitrogen at a rate of 10° C./min, up to a final temperature of 350° C. Nickel and Alumel™ were used as the calibration standards.

### MSA

MSA analyses were performed using a VTI SGA-100 Vapor Sorption Analyzer. Sorption and desorption data were collected over a range of 5% to 95% relative humidity (RH) at 10% RH intervals under a nitrogen purge. Samples were not dried prior to analysis. Equilibrium criteria used for analysis were less than 0.0100% weight change in 5 minutes, with a maximum equilibration time of 3 hours if the weight criterion was not met. Data were not corrected for the initial moisture content of the samples. NaCl and PVP were used as calibration standards.

### Example 1. Preparation of Form I

Polymorphic Form I of Compound I was obtained by slurrying of Compound I in ethyl acetate or methanol at room temperature, or in a mixture IPA:water 1:1 at ˜58° C. Additionally, Form I was obtained by slow cooling of a solution of Compound I in acetonitrile. Form I remained stable as a solid form when stressed at ˜94% RH for 10 days.

Form I was analyzed by XRPD, DSC, TGA, and MSA. FIG. 1A shows an XRPD pattern of Form I. FIG. 1B shows a DSC graph of Form I. As shown in the DSC graph, an endotherm onset at about 215.5° C. was observed. FIG. 1C shows a TGA graph of Form I. As shown in the TGA graph, no weight loss was observed below about 213.0° C. FIG. 1D shows a MSA graph of Form I. Moisture sorption data shows ˜0.1 wt % loss upon equilibration at ˜5% RH, and ˜0.4 wt % gain between ˜5% and ˜95% RH. A ˜0.4 wt % loss occurred between ˜95% RH and ˜5% RH, with a small hysteresis between sorption and desorption steps. Overall, the data shows that Form I has low hygroscopicity.

### Example 2. Preparation of Form II

Polymorphic Form II was obtained by slurrying of Compound I in acetone or acetonitrile at room temperature, and in ethyl acetate or acetonitrile at an elevated temperature. Additionally, Form II was obtained by slow cooling or slow evaporation of solutions in a variety of solvents or solvent mixtures.

Form II was analyzed by XRPD, DSC, TGA, and MSA. FIG. 2A shows an XRPD pattern of Form II. FIG. 2B shows a DSC graph of Form II. As shown in the DSC graph, an endotherm onset at about 206.7° C. (peak maximum) was observed. FIG. 2C shows a TGA graph of Form II. As shown in the TGA graph, no weight loss was observed below about 202.3° C. FIG. 1D shows a MSA graph of Form I. Moisture sorption data shows ˜0.6 wt % loss upon equilibration at ˜5% RH, negligible weight change between ˜5% and ˜95% RH, and negligible weight change between ˜95% RH and ˜5% RH. Overall, the data shows that Form II has low hygroscopicity.

### Example 3. Preparation of Form III

Polymorphic Form III was obtained by vapor diffusing a solution of Compound I in a THF/diethyl ether solvent system. Form III was analyzed by XRPD and DSC. FIG. 3A shows an XRPD pattern of Form III. FIG. 3B shows a DSC graph of Form III.

### Example 4. Preparation of Form IV

Polymorphic Form IV was obtained by slow cooling a solution of Compound I in a mixture of methanol and water. Form IV was analyzed by XRPD and DSC. FIG. 4A shows an XRPD pattern of Form IV. FIG. 4B shows a DSC graph of Form IV.

### Example 5. Preparation of Form V

Polymorphic Form V was obtained by vapor diffusing a solution of Compound I in a THF/hexane solvent system. Form V was analyzed by XRPD and DSC. FIG. 5A shows an XRPD pattern of Form V. FIG. 5B shows a DSC graph of Form V.

### Example 6. Preparation of Form VI

Polymorphic Form VI was obtained by slow evaporating a solution of Compound I in an acetone/acetonitrile solvent system. Form VI was analyzed by XRPD and DSC. FIG. 6A shows an XRPD pattern of Form VI. FIG. 6B shows a DSC graph of Form VI.

### Example 7. Preparation of Form VII

Polymorphic Form VII was obtained by spontaneous crystallization from a solution of Compound I in chloroform. Form VII was analyzed by XRPD and DSC. FIG. 7A shows an XRPD pattern of Form VII. FIG. 7B shows a DSC graph of Form VII.

### Example 8. Slurry Interconversion

Slurry interconversion experiments were performed at temperatures ranging from room temperature to ˜81° C. Solvent systems used for the study were nitromethane, acetonitrile, and a THF/heptane 1:2 (v/v) mixture. All experiments yielded Form II, suggesting that this form is the most stable form at ambient temperature and elevated temperature up to ˜81° C. The results are summarized in Table 8.

TABLE 8

| Starting Forms | Conditions | XRPD Result |
|---|---|---|
| I, II, III, IV, V, VI, VII | Nitromethane, RT, | Form II |
| (~5 mg each)′ | 3 days | |
| | Nitromethane, ~78-81° C., | Form II |
| | 3 days | |
| | ACN, RT, 1 day | Form II |
| I, II, III, V | ACN, ~66-69° C., | Form II |
| (~5 mg each) | 1 day | |
| I, II, VI | THF/heptane 1:2, ~61° C., | Form II |
| (~15 mg each) | 1 day | |
| | THF:heptane 1:2, | Form II |
| | RT, 1 day | |

All documents, including patents, patent application and publications cited herein, including all documents cited therein, tables, and drawings, are hereby expressly incorporated by reference in their entirety for all purposes.

While the foregoing written description of the polymorphic forms, uses, and methods described herein enables one of ordinary skill in the art to make and use the polymorphic forms, uses, and methods described herein, those of ordinary skill in the art will understand and appreciate the existence of variations, combinations, and equivalents of the specific embodiments, methods, and examples herein.

1. (canceled)

2. A polymorph of a compound of the formula: 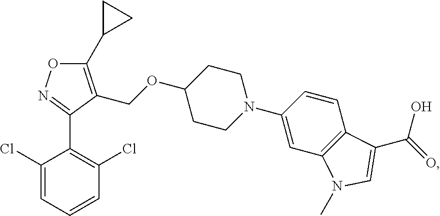 [SMILES: Cn1cc(C(=O)O)c2ccc(N3CCC(OCc4c(-c5c(Cl)cccc5Cl)noc4C4CC4)CC3)cc21]
characterized as having an X-Ray Powder Diffraction (XRPD) pattern comprising peaks at angles 2-theta of 14.40±0.20, 20.48±0.20, and 24.74±0.20 degrees.

3. The polymorph of claim 2, characterized as having an XRPD pattern comprising peaks at angles 2-theta of 14.40±0.20, 15.51±0.20, 19.20±0.20, 20.48±0.20, and 24.74±0.20 degrees.

4. The polymorph of claim 2, characterized as having an XRPD pattern substantially as shown in FIG. 1A.

5. The polymorph of claim 2, characterized as having a Differential Scanning calorimetry (DSC) graph comprising an endotherm onset at about 215.5° C.

6. The polymorph of claim 2, characterized as having a DSC graph substantially as shown in FIG. 1B.

7. The polymorph of claim 2, characterized as having a Thermogravimetric Analysis (TGA) graph comprising no weight loss below about 213.0° C.

8. The polymorph of claim 2, characterized as having a TGA graph substantially as shown in FIG. 1C.

9. The polymorph of claim 2, characterized as having a Moisture Sorption Analysis (MSA) graph substantially as shown in FIG. 1D.

10-36. (canceled)

37. A method of preparing the polymorph of claim 2, comprising slurrying a solution comprising the compound and a solvent, wherein the solvent comprises methanol, ethyl acetate, or a mixture of isopropanol and water.

38. A method of preparing the polymorph of claim 2, comprising slow cooling a solution comprising the compound and a solvent, wherein the solvent comprises acetonitrile.

39. The method of claim 37, wherein the solvent comprises methanol.

40. The method of claim 37, wherein the solvent comprises ethyl acetate.

41. The method of claim 37, wherein the solvent comprises a mixture of isopropanol and water.

42. The method of claim 41, wherein the solvent comprises a 1:1 mixture of isopropanol and water.

43-45. (canceled)

46. A pharmaceutical composition comprising the polymorph of claim 2, and a pharmaceutically acceptable carrier.

47. A method of treating a liver disorder in a subject in need thereof, comprising administering a therapeutically effective amount of the polymorph of claim 2.

48. The method of claim 47, wherein the liver disorder is liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), or non-alcoholic steatohepatitis (NASH).

49-50. (canceled)

51. The method of claim 48, wherein the liver disorder is NASH.

52. The pharmaceutical composition of claim 46, wherein the polymorph is characterized as having an XRPD pattern comprising peaks at angles 2-theta of 14.40±0.20, 15.51±0.20, 19.20±0.20, 20.48±0.20, and 24.74±0.20 degrees.

53. The method of claim 51, wherein the polymorph is characterized as having an XRPD pattern comprising peaks at angles 2-theta of 14.40±0.20, 15.51±0.20, 19.20±0.20, 20.48±0.20, and 24.74±0.20 degrees.
